(19) United States
(12) Patent Application Publication
(10) Pub. No.: US 2009/0125080 A1
(43) Pub. Date: May 14, 2009

(54) INTRAVENTRICULAR ELECTRODES FOR ELECTRICAL STIMULATION OF THE BRAIN

(76) Inventor: Erwin B. Montgomery, JR., Middleton, WI (US)

Correspondence Address: Erwin B. Montgomery, Jr., 3418 John Muir Dr., Middleton, WI, 53562, US

(21) Appl. No.: 11/938,591

(22) Filed: Nov. 12, 2007

(51) Int. Cl. A61N 1/05

(52) U.S. Cl. 607/45

(57) ABSTRACT

An electrode—preferably an anode (current sink)—is implanted within a ventricle of the brain so that the cerebrospinal fluid therein, which is highly conductive, effectively makes the ventricle a conductive extension of the anode. An opposing electrode (i.e., a cathode) can then be situated within or outside the brain (e.g., extradurally) so that a portion of the brain to be electrically stimulated is situated between the electrodes. The electrodes can then be energized at appropriate frequencies and current/voltage levels to apply the desired stimulation, in a manner similar to preexisting Deep Brain Stimulation (DBS), Extradural Motor Cortex Stimulation (EMCS), and other electrical brain stimulation procedures.

### FIELD OF THE INVENTION

This document concerns an invention relating generally to methods and devices for electrical stimulation of the brain.

### BACKGROUND OF THE INVENTION

Electrical stimulation of the brain is being studied for the treatment of a wide variety of neurological and psychiatric conditions. An example of a first arrangement, known as Extradural Motor Cortex Stimulation (EMCS), is illustrated in simplified form in FIG. 1A. Here, a current source (i.e., a cathode) 100 is situated extradurally (i.e., atop or outside the dura mater 10), with a nearby current sink (i.e., an anode) 102 also being extradurally situated nearby. Respective leads 104 and 106 extend from each of the current source 100 and current sink 102 through the skull 12, usually through a burr cap (not shown), i.e., a “plug” inserted through a hole cut in the skull 12. The leads 104 and 106 are then usually run subcutaneously down the skull 12 and neck to a power supply 108, e.g., a neurostimulator subcutaneously implanted near the chest. The power supply 108 is programmable to provide current—usually in timed pulses having some desired magnitude—to the current source 100, with the current then flowing through the dura 10, the subarachnoid space 14 (i.e., the space between the dura 10 and the brain 16), and then into the adjacent area of the brain 16 between the current source 100 and current sink 102 (with current flow being schematically illustrated in phantom lines in FIG. 1A). EMCS has proven to be effective in treating some cases of Parkinson's disease, and is under investigation for treatment of conditions such as epilepsy, depression, and obsessive compulsive disorder. However, the effectiveness of EMCS is limited, possibly because the emitted current can only stimulate portions of the cortex 16 near the cranium 12 owing to the extradural locations of the current source 100 and current sink 102. In this respect, it is believed that effectiveness of EMCS is also limited because at least a portion of the current is shunted from the current source 100 to the current sink 102 by the conductive cerebrospinal fluid in the subarachnoid space 14, with only a limited amount of current entering the cortex 16.

Another arrangement for electrostimulation of the brain 16, known as Deep Brain Stimulation (DBS), is illustrated in simplified form in FIG. 1B. In common DBS arrangements, an elongated rigid lead 150 bears one or more current sources (cathodes) 152, and also bears one or more current sinks (anodes) 154 spaced from the current sources 152 along the length of the lead 150. In the arrangement of FIG. 1B, several current sources 152 are shown spaced along the length of the lead 150, and a single current sink/anode 154 is shown at the end of the lead 150. The lead 150 is inserted through the skull 12—again usually through a burr cap (not shown) installed in a hole in the skull 12—and through the dura 10 and subarachnoid space 14, and finally into the brain 16 to situate the electrodes 152 and 154 adjacent to or within the area of the brain 16 to be electrically stimulated (often the subthalamic nucleus and/or globus pallidus interna). Supplemental leads 156 and 158 then extend from the electrodes 152 and 154 (usually subcutaneously) to a power supply 160 (also usually located subcutaneously), which supplies the current sources 152 with current to generate current flow patterns such as those shown in phantom lines in FIG. 1B. DBS can be highly effective, but since the installation of the lead 150 is more invasive, the implementation of DBS bears greater risks (such as the risk of hemorrhaging caused by insertion of the lead 150). Additionally, the effectiveness of DBS can decrease, and/or adverse side effects can arise, if the lead 150 is not ideally situated. This sometimes occurs since the lead 150 is usually inserted in reliance on a stereotactic map of the brain generated by imaging techniques (such as Computed Tomography (CT) and/or Magnetic Resonance Imaging (MRI)), and the difficulties involved with referencing the map versus the actual brain 16 causes challenges with proper location of the lead 150.

### SUMMARY OF THE INVENTION

The invention involves systems and methods for brain stimulation which are intended to at least partially solve the aforementioned problems. To give the reader a basic understanding of some of the advantageous features of the invention, following is a brief summary of preferred versions, with reference being made to FIGS. 2A and 2B of the accompanying drawings to enhance the reader's understanding. Since this is merely a summary, it should be understood that more details regarding the preferred versions of the invention may be found in the Detailed Description set forth elsewhere in this document. The claims set forth at the end of this document then define the various versions of the invention in which exclusive rights are secured.

The brain is stimulated by a current source (i.e., a cathode) and a current sink (i.e., an anode) wherein one of the current source and the current sink is situated within a ventricle of the brain, and the other of the current source and the current sink is situated outside of the ventricle, such that a portion of the brain which is to be subjected to electrical stimulation is situated between the current source and the current sink. Since the ventricles are filled with (relatively) highly conductive cerebrospinal fluid, and the fluid within the ventricle bearing the current sink (or source) is in conductive communication with the current sink (or source), the ventricle effectively becomes a conductive extension of the current sink (or source). Thus, a ventricle bearing the current sink will draw current flow from the current source to the ventricle, and subsequently to the current sink and any associated lead. Conversely, a ventricle bearing the current source will emit current toward the current sink and any associated lead.

An exemplary arrangement of this nature is schematically depicted in FIG. 2A, wherein a current sink (anode) 202 is situated within one of the lateral ventricles 18, and a current source (cathode) 200 is spaced from the ventricle 18 across the portion of the brain 16 to be stimulated. The current source 200 is preferably situated extradurally (i.e., outside and above the dura mater 10), though it can be situated within the brain 16 instead (as will be discussed later in this document, e.g., with respect to FIG. 2B). The current source 200 is connected to (or may be formed of) an elongated lead 204 which preferably runs subcutaneously to a power supply 208, such as an subcutaneous neurostimulator implanted near the chest. The current sink 202 is preferably similarly connected to or formed from an elongated lead 206 extending from the ventricle 18 through the cortex 16, subarachnoid space 14, dura mater 10, and skull 12, with the lead 206 (or a connecting lead) then preferably running subcutaneously to the positive terminal of the power supply 208.

The lead 206 forming the current sink 202 preferably has a conductive core 210 (or other conductive path) which bears an outer insulating coating 212 along its length, save for the location(s) along the lead 206 which are to define the electrode(s) for electrically communicating with the cerebrospinal fluid within the ventricle 18. In the exemplary lead of FIG. 2A, the insulating coating 212 is omitted at the end of the lead 206 to expose the conductor 210 and thereby define the current sink 202. Insulating the exterior length of the current sink lead 206 helps to better direct current from the current source 200 solely to the ventricle 18, and to prevent the current from “short-circuiting” along different paths, such as to the cerebrospinal fluid within the subarachnoid space 14, and/or to portions of the brain 16 located directly between the current source 200 and the length of the lead 206 of the current sink 202. Versions of the invention which generate current flow between an extradural current source 200 and a ventricular current sink 202 may be particularly useful because most axons within the brain 16 are believed to be aligned roughly parallel to the surface of the brain 16, and therefore current flow between an extradural current source 200 and a ventricular current sink 202 will be oriented generally perpendicularly to the axons, which may result in more effective activation of neurons.

To assist in an operating physician's installation of the current sink 202 within a selected ventricle 18, its lead 206 preferably has an interior passage 214 extending along its length so that when the lead 206 is inserted within a patient's skull 12 and it reaches the ventricle 18, cerebrospinal fluid can be communicated through the interior passage 214 to the operating physician to indicate that the ventricle 18 has been reached. As an example of such an arrangement, the exemplary lead 206 of FIG. 2A has a form similar to a conductive hollow needle 210 with an insulating covering 212, with the insulating covering 212 being removed at the tip to define the current sink 202 on the lead 206.

The opposing current source 200 and its lead 204, if not installed within a ventricle 18, can have a conventional form. Thus, it might be formed as in FIG. 2A, as an electrode 200 affixed to a wire 204 having a solid cross-section with an insulated exterior. The current source 200 might simply take the form of an area on the lead 204 at which insulation is removed to define the current source 200.

Arrangements of this nature may be used in combination with a DBS electrode, e.g., to correct the use of a previously-implanted DBS electrode which is not providing the desired effect. As an example, the lead 150 of FIG. 1B might be implanted so that the current sources 152 and sinks 154 are at or near the subthalamic nucleus (as desired), but if implanted too near the internal capsule, some of the current may “bleed over” to the internal capsule and cause intolerable tonic contraction of the patient's muscles. As a result, the patient may prefer to simply leave the DBS lead 150 inactive. FIG. 2B then illustrates the use of a ventricular electrode to “salvage” the use of an implanted DBS lead 250. Here, a lead 262 similar to the lead 206 of FIG. 2A is installed in the brain 16 with its current sink 264 situated within the ventricle 18, thereby effectively converting the entire ventricle 18 into a current sink. The current sink 264 is connected to the positive terminal of the power supply 260 in lieu of the current sink 254 of the DBS lead 250, with only the current source(s) 252 of the lead 250 being active. As a result, current supplied to the current source(s) 252 flows toward the ventricle 18, and to the current sink 264 therein, thereby attracting the emitted current to the ventricle 18 in a medial direction, and away from the internal capsule, so that side effects are reduced or eliminated. In similar respects, the intraventricular current sink 202 of FIG. 2A might be installed to adapt the performance of an ineffective extradural current sink 102 as in FIG. 1A.

Further advantages, features, and objects of the invention will be apparent from the remainder of this document in conjunction with the associated drawings.

### BRIEF DESCRIPTION OF THE DRAWINGS

FIG. 1A is a simplified side elevational cross-sectional view (i.e., a sagittal view) of a human head shown with a conventional Extradural Motor Cortex Stimulation (EMCS) system installed, wherein a current source (cathode) 100 and current sink (anode) 102 are situated in spaced relationship atop the dura mater 10. Current supplied from the power supply 108 to the current source 100 flows at least partially into the adjacent cortex 16 as it travels to the current sink 102, thereby stimulating neurons within the cortex 16.

FIG. 1B is a simplified front elevational cross-sectional view (i.e., a coronal view) of a human head shown with a conventional Deep Brain Stimulation (DBS) system installed, wherein a lead 150 bearing several connected current sources (cathodes) 152 emit current toward a current sink (anode) 154 which is also situated on the lead 150, thereby electrically stimulating regions of the brain 16 adjacent to and between the current sources 152 and current sink 154.

FIG. 2A is a simplified sagittal view of a human head shown with an intraventricular current sink 202 installed, wherein the conductivity of the cerebrospinal fluid within the ventricle 18 effectively makes the entire ventricle a conductive extension of the current sink 202, whereby current travels from the current source 200 to the ventricle 18 and stimulates the portions of the brain 16 therebetween.

FIG. 2B is a simplified coronal view of a human head shown with an intraventricular current sink 264 being used in conjunction with a current source 252 from a conventional DBS lead 250, whereby current is drawn from the current source 252 to the conductive ventricle 18, thereby stimulating portions of the brain 16 therebetween.

### DETAILED DESCRIPTION OF PREFERRED VERSIONS OF THE INVENTION

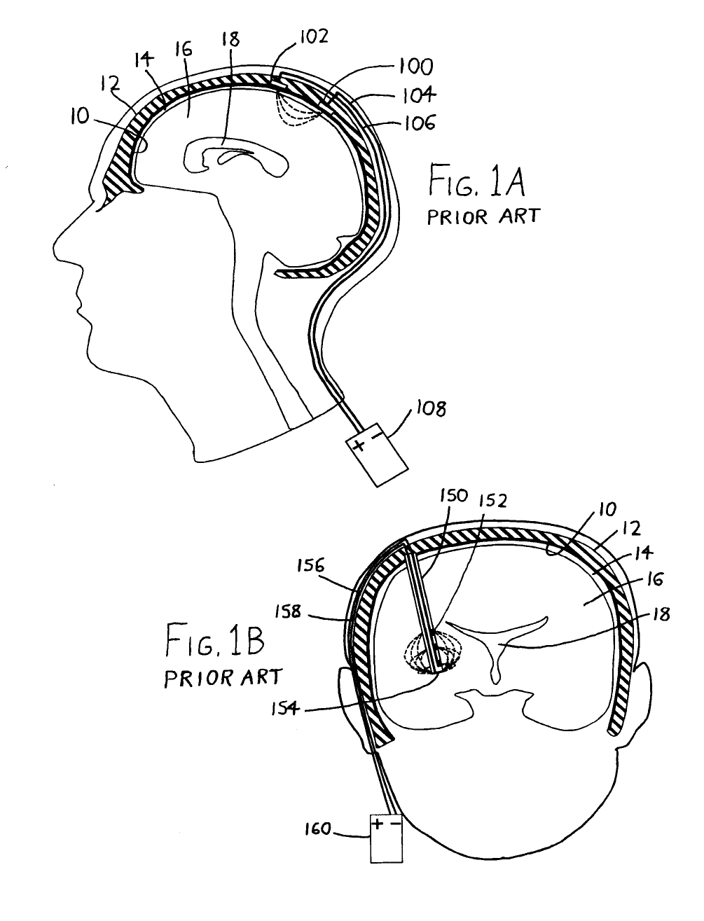

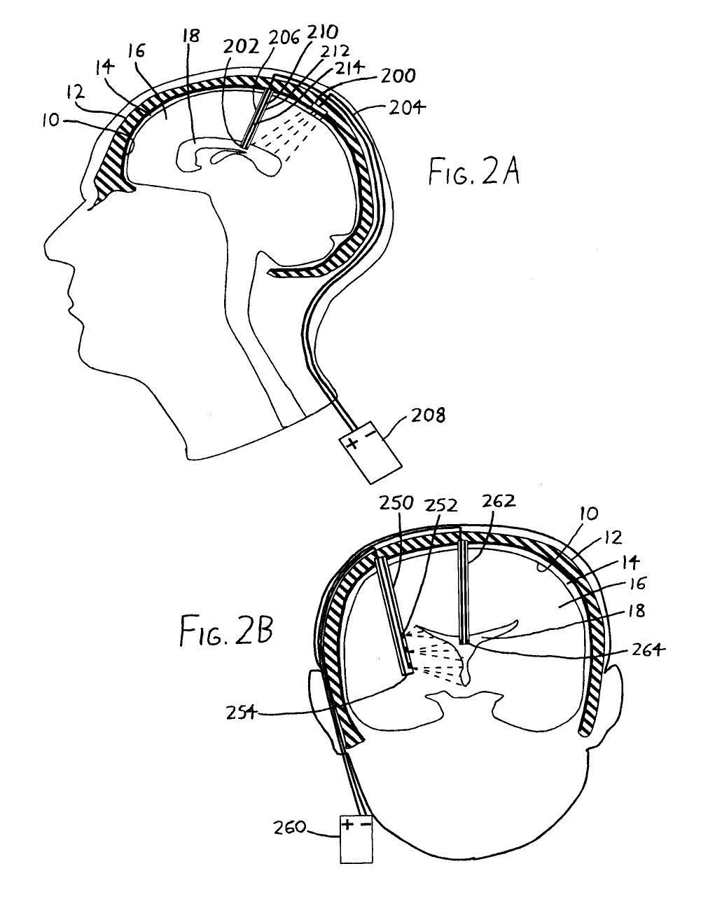

To elaborate on the discussion in the Summary above, the invention may be implemented instead of preexisting brain stimulation systems, or it may be implemented alongside them. As an example, in FIG. 2B, the ventricular current sink 264 might be used in addition to the current sink 254, rather than instead of it. In this case, the current sinks 254 and 264 might operate in tandem (e.g., with both being connected to the positive terminal of the power supply 260), or one might be operated differently than the other (e.g., they could be connected to separate terminals which operate them at different voltages, frequencies, etc. so that current flow between the current source 252 and the sink 254 differs from that between the source 252 and the sink 264). In similar respects, the arrangement of FIG. 2A might utilize an extradural current sink (as in FIG. 1A) in addition to the intraventricular current sink 202. It is also possible to provide the current source(s) and current sink(s) on the same lead, and to situate one of them within a ventricle. As an example, in FIG. 2B, the lead 262 might be replaced with a lead similar to lead 250, with its current sink 254 situated within a ventricle and the current sources 252 situated such that the brain's region of interest for stimulation is situated between the current sources 252 and the ventricle.

The foregoing Summary generally discussed placing a current sink within a ventricle, and situating a current source either extradurally or within the brain. It is instead possible to situate the current source within a ventricle, and situating the current sink on the opposite side of the region of the brain to be stimulated. However, it is believed that stimulation may be more accurately delivered to the region of interest if the current source is more closely adjacent this region, and thus it is generally preferred that the current sink (rather than the current source) be situated within a ventricle.

It is expected that the invention may be most easily implemented by modifying existing brain stimulation systems and procedures, including the ACTIVA DBS system (Medtronic Corporation, Minneapolis, Minn., USA); the RENOVA EMCS system (Northstar Neuroscience, Seattle, Wash., USA); and/or systems and procedures developed by other entities in the field, such as NeuroPace, Inc. (Mountain View, Calif., USA), Advanced Bionics Corporation (Sylmar, Calif., USA), Alpha Omega Engineering Ltd. (Nazareth Elite, Israel), and others. However, the invention might be manufactured and implemented without resort to preexisting systems.

It is notable that the invention may be implemented by use of existing power supplies (neurostimulators) by simply connecting the current source(s) and current sink(s) to the appropriate terminals of the power supplies. This is advantageous because the personnel programming the power supply, who are probably familiar with the power supplies already available in the field, need not learn to program and operate a new type of power supply. Since most power supplies utilize plug-in connections to connect the leads of selected current sources and current sinks to their appropriate terminals, a power supply will generally not need to be rewired to accommodate a separate lead for an intraventricular current sink (or current source). Instead, an appropriate adapter jack can be developed which routes power as desired. As an example, in the arrangements of FIGS. 1A and 1B, the leads 104 and 106, and 156 and 158, are generally bundled and terminate in a connecting jack which plugs into a single port in the power supplies 108 and 160. It is therefore a relatively simple matter to construct an adapter jack which, for example, plugs into the power supply 260 of FIG. 2B, and branches into a negative port to which the lead 250 of the current source 252 may be connected, and into a positive port to which the lead 262 of the current sink 264 may be connected.

It is also notable that the current sinks 202 (FIG. 2A) and 264 (FIG. 2B) and their associated leads 206 and 262 may simply take the form of the current sink 154 (FIG. 1B) and lead 150 of a conventional DBS arrangement, in which case the electrodes of the current sources 152 might be left unused. However, as previously noted, the invention usefully incorporates a lead having an internal passage—e.g., the internal passage 214 of the lead 206 of FIG. 2A—so that the operating physician is better able to determine when the lead 206 reaches a ventricle. As was also noted previously, placement of leads can be difficult, and it is highly desirable to avoid erroneous lead insertions. If such erroneous insertions are detected during the course of an operation, they may require withdrawal and reinsertion of a lead, which runs greater risk of complications. By providing a passage 214 along the length of the lead 206, and more particularly from a region on the lead 206 at or adjacent the current source 202 to a region on the lead 206 which will rest outside the brain 16 during and after the installation operation, an operating physician can determine when the lead 206 has reached a ventricle 18 by watching for the expression of cerebrospinal fluid at the exit of the passage 214.

The lead 206 of FIG. 2A (and the lead 262 of FIG. 2B, which is illustrated with a similar configuration) may take forms other than the one shown, wherein the internal passage 214 is defined within the length of a conductor 210 leading to the current sink 202, with an insulating coating 212 shielding the conductor 210 save for at the current sink 202. As an example, the lead 206 might be formed of a rigid tubular insulator which is inserted within the brain until cerebrospinal fluid is detected at its passage exit. The conductor might then simply take the form of a solid conductive wire which is inserted within the tubular insulator to provide the current sink.

A benefit of the invention is that since the ventricles are located (roughly) in the middle of the brain, the invention tends to direct current flow in a medial direction from a current source. Most of the structures of the brain which are of interest for (currently known) electrical stimulation procedures are generally centrally located within the brain, and thus the use of a ventricular electrode, in conjunction with opposing extradural and/or implanted brain electrodes, can help to better direct current to the desired structures.

The invention is not intended to be limited to the preferred versions of the invention described above, but rather is intended to be limited only by the claims set out below. Thus, the invention encompasses all different versions that fall literally or equivalently within the scope of these claims.

What is claimed is:

1. A system for stimulating brain cells including a current source and a current sink, wherein
a. one of the current source and the current sink is within a ventricle of the cerebrum of a brain, and
b. the other of the current source and the current sink is outside of the ventricle,
with at least a portion of the cerebrum being situated between the current source and the current sink.

2. The system of claim 1 wherein the current source and current sink are situated on separate spaced leads.

3. The system of claim 1 wherein the current sink is located within the ventricle.

4. The system of claim 1 wherein the one of the current source and the current sink located outside of the ventricle is located outside of a dura mater surrounding the brain.

5. The system of claim 4 wherein the one of the current source and the current sink located outside of the dura mater is the current source.

6. The system of claim 5 further including a power supply located outside of a skull wherein the brain is located, and wherein the current source is connected by a lead to the power supply.

7. The system of claim 1 wherein the one of the current source and the current sink located within the ventricle is situated on an elongated lead extending through:
a. a skull situated about the cerebrum,
b. a dura mater situated beneath the skull and about the cerebrum,
c. a subarachnoid space situated beneath the dura mater and about the cerebrum, and
d. at least a portion of the cerebrum,
and wherein the elongated lead includes an interior passage therein, the interior passage extending through the skull, dura mater, subarachnoid space, and cerebrum to open onto the ventricle, whereby cerebrospinal fluid within the ventricle flows through the interior passage and outside the skull upon insertion of the elongated lead within the ventricle.

8. The system of claim 7 wherein the current sink is located within the ventricle.

9. The system of claim 7 wherein the one of the current source and the current sink located within the ventricle is connected to a conductive path extending along the elongated lead through the cerebrum, subarachnoid space, dura mater, and skull.

10. The system of claim 9 wherein the elongated lead includes an insulating layer between the conductive path and:
a. the subarachnoid space, and
b. the cerebrum.

11. A method for stimulating brain cells including the step of applying current between a current source and a current sink, wherein:
a. one of the current source and the current sink is within a ventricle of the brain, and
b. the other of the current source and the current sink is outside of the ventricle.

12. The method of claim 11 wherein:
a. the ventricle is located within a cerebrum, and
b. the cerebrum is situated between the current source and the current sink, with the applied current flowing through the cerebrum.

13. The method of claim 11 wherein the current sink is within the ventricle.

14. The method of claim 13 wherein the current sink is located on an elongated lead extending through:
a. a skull situated about the brain,
b. a dura mater situated beneath the skull and about the brain,
c. a subarachnoid space situated beneath the dura mater and about the brain, and
d. a cerebrum beneath the subarachnoid space,
wherein the ventricle is located within the cerebrum.

15. The method of claim 14 wherein the current source is situated outside the dura mater, with the dura mater being situated between the current source and the subarachnoid space.

16. The method of claim 11 wherein the current source is outside of the ventricle, and is located outside a dura mater situated beneath the skull and about the brain.

17. The method of claim 11:
a. wherein:
(1) the one of the current source and the current sink within the ventricle is located on an elongated lead extending through:
(a) a skull situated about the brain,
(b) a dura mater situated beneath the skull and about the brain,
(c) a subarachnoid space situated beneath the dura mater and about the brain, and
(d) a cerebrum beneath the subarachnoid space, wherein the ventricle is located within the cerebrum;
(2) the elongated lead has an interior passage along its length and opening into the ventricle;
b. further including the step of inserting the elongated lead through the skull and into the ventricle until cerebrospinal fluid from the ventricle is received outside the skull from the interior passage.

18. The method of claim 11 wherein:
a. the one of the current source and the current sink within the ventricle includes:
(1) a conductive path extending from the ventricle to the exterior of a skull situated about the brain, and
(2) a passage extending from the ventricle to the exterior of a skull situated about the brain, whereby cerebrospinal fluid is communicated from the ventricle through the passage to the exterior of the skull;
b. the one of the current source and the current sink outside the ventricle has a solid cross-section, whereby no fluid can flow therein.

19. The method of claim 11 wherein the one of the current source and the current sink within the ventricle is located on an elongated lead extending through:
a. a skull situated about the brain,
b. a dura mater situated beneath the skull and about the brain,
c. a subarachnoid space situated beneath the dura mater and about the brain, and
d. a cerebrum beneath the subarachnoid space wherein the ventricle is located, wherein the elongated lead has an insulated exterior where the lead extends through the subarachnoid space and the cerebrum.

20. A system for stimulating brain cells including:
a. an elongated first lead including:
(1) a conductive path extending along the length of the first lead,
(2) an insulating layer situated about the conductive path,
(3) a current sink at which the insulating layer is absent, with the conductive path being exposed along the exterior of the first lead,
(4) an interior passage through which cerebrospinal fluid may flow, the interior passage extending alongside the conductive path;
b. a second lead which is separate and spaced from the first lead, the second lead extending between a power supply and an exposed current source;
wherein the current source and current sink, when situated in spaced relationship within a conductive medium, allow current flow from the current source of the second lead to the current sink of the first lead through the conductive medium.

21. The system of claim 20 wherein:
a. the first lead extends through:
(1) a skull situated about a brain,
(2) a dura mater situated about the brain beneath the skull,
(3) a subarachnoid space situated about the brain beneath the dura mater, and
(4) a cerebrum within the brain, the cerebrum having a ventricle therein,
b. the current sink of the first lead is situated within the ventricle;
c. the current source of the second lead is situated outside the dura mater, with the dura mater being situated between the current source and the subarachnoid space; and
d. the power supply of the second lead is situated outside the skull, with the skull being situated between the power supply and the dura mater.
